(19) United States
(12) Patent Application Publication
(10) Pub. No.: US 2024/0100213 A1
(43) Pub. Date: Mar. 28, 2024

(54) Composition

(71) Applicant: Reckitt & Colman (Overseas) Hygiene Home Limited, Slough, Berkshire (GB)

(72) Inventors: Nikki COLLINSON, Montvale, NJ (US); Alexandria Luz JIMENEZ, Montvale, NJ (US); Guillaume KENSICHER, Hull (GB); Charlotte WESTLEY, Hull (GB)

(21) Appl. No.: 17/768,965

(22) PCT Filed: Nov. 5, 2020
(86) PCT No.: PCT/EP2020/081206

§ 371 Date: Apr. 14, 2022

(30) Foreign Application Priority Data
Nov. 5, 2019 (GB) 1916058.9

(51) Int. Cl. A61L 9/01; A61L 9/14

(52) U.S. Cl. CPC A61L 9/01; A61L 9/14; A61L 2209/11; A61L 2209/134

(57) ABSTRACT

The present invention relates to a composition comprising a) at least 10 wt. % of at least one C1 to C4 monohydric alcohol; b) at least 20 wt.% of at least one solvent selected from the group consisting of solketal, acetone, methylpropyl ketone, dipropyl ketone, dimethyl sulfoxide, glycerine carbonate, propylene carbonate, butylene carbonate, glycerine formal, 2-ethyl hexanol, 2-butyl octanol, 2- hexyl decanol, 2-octyl dodecanol, and mixtures thereof; c) between 0.001 wt. % and 5 wt. % of at least one active constituent selected from the group consisting of fragrances, air-sanitisers, deodorizers, odor eliminators, malodor counteractants, insecticides, insect repellents, medicinal substances, disinfectants, aroma therapy compositions and combinations thereof; and d) water. The invention further relates to a device comprising a container and a dispenser, wherein the container contains the composition according to the invention. The invention also relates to a use of the composition according to the invention for freshening or fragrancing air.

The present invention relates to a composition comprising an active constituent, particularly a fragrance, to a device comprising a container comprising said composition and to the use of said composition for freshening or fragrancing air.

### BACKGROUND

Products comprising an active constituent are provided in several formats. For example, products having an active constituent such as fragrances, deodorizers, odor eliminators, malodor counteractants, and aroma therapy compositions, can be provided in a variety of forms. These include scented candles, wax melters, reed stick emanators, scented gels, liquid electrical plugins and aerosols. Of these aerosols are probably the most widely used air freshening product. The reason for their popularity no doubt stems from their ease of use (portable and requires no power source), rapid effect (the product is dispensed and dispersed immediately into the air), safety (no need for either electrical power or combustion) and simplicity.

However, aerosols require the use of propellants. The two main types of propellants used in aerosol dispensers are liquefied gas propellants, such as hydrocarbon propellants, hydrofluorocarbon propellants and compressed gas propellants. Propellants contain Volatile Organic Compounds (VOCs). This means that propellants, besides being expensive, are environmentally damaging.

Formulations having an active constituent, and which are provided as a sprayable composition, can be used together with a device that sprays said formulation automatically, for example every certain amount of time. In this case, the device is maintained in the same spot for several days/weeks where it will spray the formulation. This brings the problem of potential fallout and residue on surfaces. If the formula is an aerosol it benefits from high content of Volatile Organic Compounds (VOC)—up to 70% in weight—and can therefore have enough volatile material for the residue not to happen; and if the active constituent is a fragrance, enough solvent to solubilize the fragrance.

Furthermore, when the formula is not in an aerosol format, the amount of VOC is less, meaning that if e.g. the active constituent is a fragrance, there is not enough solvent to solubilize the fragrance. Normally, this is solved by adding surfactants to the formulation to stabilize the active constituent, e.g. the fragrance, in solution. These surfactants are mainly solids and re-crystallize once they fall back on the floor.

Therefore, there is a need of a non-aerosol sprayable composition that after being sprayed does not fallout and does not create residues on surfaces. With other words, there is a need of a sprayable composition, said composition being stable and having a solubilized active constituent and being capable of being sprayed without falling out and without creating residues on surfaces.

It is an object of the present invention to provide a composition comprising an active constituent, preferably a fragrance, wherein said composition is sprayable and once sprayed does not fall out and does not create residues on surfaces.

It is an object of the present invention to provide a device comprising a container and a non-aerosol, spray generation dispenser, wherein said device can spray the composition according to the invention.

### SUMMARY OF THE INVENTION

In a first aspect, the present invention relates to a composition comprising:
a. at least 10 wt. % of at least one C1 to C4 monohydric alcohol, based on the total weight of the composition;
b. at least 20 wt. % of at least one solvent selected from the group consisting of solketal, acetone, methylpropyl ketone, dipropyl ketone, dimethyl sulfoxide, glycerine carbonate, propylene carbonate, butylene carbonate, glycerine formal, 2-ethyl hexanol, 2-butyl octanol, 2- hexyl decanol, 2-octyl dodecanol, and mixtures thereof, based on the total weight of the composition;
c. between 0.001 wt. % and 5 wt. % of at least one active constituent, based on the total weight of the composition, wherein the active constituent is selected from the group consisting of fragrances, air-sanitisers, deodorizers, odor eliminators, malodor counteractants, insecticides, insect repellents, medicinal substances, disinfectants, aroma therapy compositions and combinations thereof; and
d. water.

In one embodiment, said composition is free of propellants.

The at least one C1 to C4 monohydric alcohol may be selected from the group consisting of ethanol, isopropyl alcohol and combinations thereof; preferably said at least one C1 to C4 monohydric alcohol is isopropyl alcohol. The composition may comprise at least 12 wt. % of said at least C1 to C4 monohydric alcohol, based on the total weight of the composition, preferably at least 15 wt. %, more preferably between 15 wt. % and 50 wt. %, even more preferably between 15 wt. % and 30 wt. %.

The at least one solvent may be solketal. The composition may comprise at least 25 wt. % of the at least one solvent, based on the total weight of the composition, preferably between 25 wt. % and 50 wt. %, more preferably between 25 wt. % and 40 wt. %, even more preferably between 27 wt. % and 35 wt. %.

In an embodiment, said composition comprises less than 25 wt. % volatile organic compounds, based on the total weight of the composition, preferably less than 20 wt. %, more preferably less than 18 wt. %

In an embodiment, said at least one active constituent is selected from the group consisting of fragrances, deodorizers, odor eliminators, malodor counteractants, aroma therapy compositions, and combinations thereof, preferably said at least one active constituent is a fragrance.

The composition may comprise between 0.1 wt. % and 5 wt. % of said at least one active constituent, based on the total weight of the composition, preferably between 0.5 wt. %

ad 4 wt. %, more preferably between 1 wt. % and 3 wt. %, even more preferably between 1.5 wt. % and 2.5 wt. %.

In an embodiment, said composition is free of acetone.

In an embodiment, said composition is an air freshener.

In a second aspect, the invention relates to a device comprising a container and a non-aerosol, spray generation dispenser, wherein the container contains the composition according to the invention and it attaches to the non-aerosol, spray generation dispenser.

In one embodiment, said non-aerosol, spray generation dispenser is an auto-actuable dispenser. For example, said non-aerosol, spray generation dispenser may be automatically actuated every 30 minutes, preferably every 20 minutes, more preferably every 10 minutes.

In another embodiment, said non-aerosol, spray generation dispenser is a manually-actuable dispenser.

The device may be an air-freshening or air-fragrancing device.

In a third aspect, the invention relates to a use of the composition according to the invention for freshening or fragrancing air.

### Definitions

The following definitions are used in the present application.

“C1 to C4 monohydric alcohol” as used in the present application means an organic compound with one alcohol functional group and having between 1 and 4 Carbon atoms.

“Solketal” as used in the present application means a protected form of glycerol with an isopropylidene acetal group joining two neighboring hydroxyl groups. It is also know with the following name (2,2-Dimethyl-1,3-dioxolan-4-yl)methanol.

“Volatile organic compounds (VOC)” as used in the present application means organic chemicals that have a high vapor pressure at ordinary room temperature. These chemicals are any compound of carbon, excluding carbon monoxide, carbon dioxide, carbon acid, metallic carbides, carbides and ammonium carbonate, which participates in atmospheric photochemical reactions.

“Sprayable” as used in the present application means a composition that can be dispersed as a fine mist.

“Auto-actuable dispenser” as used in the present application means a dispenser that can be actuated automatically every certain amount of time.

“Air freshener” as used in the present application means a substance or a device for making the air in a room smell fresh.

### DETAILED DESCRIPTION OF THE INVENTION

In a first aspect, the present invention relates to a composition comprising:
a. at least 10 wt. % of at least one C1 to C4 monohydric alcohol, based on the total weight of the composition;
b. at least 20 wt. % of at least one solvent selected from the group consisting of solketal, acetone, methylpropyl ketone, dipropyl ketone, dimethyl sulfoxide, glycerine carbonate, propylene carbonate, butylene carbonate, glycerine formal, 2-ethyl hexanol, 2-butyl octanol, 2- hexyl decanol, 2-octyl dodecanol, and mixtures thereof, based on the total weight of the composition;
c. between 0.001 wt. % and 5 wt. % of at least one active constituent, based on the total weight of the composition, wherein the active constituent is selected from the group consisting of fragrances, air-sanitisers, deodorizers, odor eliminators, malodor counteractants, insecticides, insect repellents, medicinal substances, disinfectants, aroma therapy compositions and combinations thereof; and
d. water.

The present inventors have observed that by combining at least one C1 to C4 monohydric alcohol and at least one solvent selected from the group consisting of solketal, acetone, methylpropyl ketone, dipropyl ketone, dimethyl sulfoxide, glycerine carbonate, propylene carbonate, butylene carbonate, glycerine formal, 2-ethyl hexanol, 2-butyl octanol, 2- hexyl decanol, 2-octyl dodecanol, and mixtures thereof, a composition is obtained, which once sprayed does not fallout and does not leave residues on surfaces. It will be understood that if the composition falls-out, then the retention of the composition in the air is reduced, which is undesirable.

The composition according to the invention can be sprayed without the need of adding propellants, therefore, the composition can be considered as being non-aerosol and preferably it is free of propellants.

The at least one C1 to C4 monohydric alcohol may be selected from the group consisting of ethanol, isopropyl alcohol and combinations thereof; preferably said at least one C1 to C4 monohydric alcohol is isopropyl alcohol.

The composition may comprise at least 12 wt. % of said at least C1 to C4 monohydric alcohol, based on the total weight of the composition, preferably at least 15 wt. %, more preferably between 15 wt. % and 50 wt. %, even more preferably between 15 wt. % and 30 wt. %.

The specially preferred at least one solvent is solketal. The composition may comprise at least 25 wt. % of the at least one solvent, based on the total weight of the composition, preferably between 25 wt. % and 50 wt. %, more preferably between 25 wt. % and 40 wt. %, even more preferably between 27 wt. % and 35 wt. %.

The present inventors have observed that the combination of solketal and isopropyl alcohol allows for the formulation of compositions comprising more than 1wt. % of an active constituent, e.g. a fragrance, wherein the composition is stable, i.e. it is present as a single phase, and can be sprayed without leaving residues on surfaces. It should be noted that these stable formulations are obtained without the need of adding any surfactant.

The composition may comprise less than 25 wt. % volatile organic compounds, based on the total weight of the composition, preferably less than 20 wt., more preferably less than 18 wt. %.

Compositions having an amount of VOC of less than 18 wt. %, based on the total weight of the composition, comply with the regulation in the USA for non-aerosol liquid/pump spray.

Even with a low content of VOC, i.e. less than 25 wt. %, the compositions described herein are stable.

The said at least one active constituent may be selected from the group consisting of fragrances, deodorizers, odor eliminators, malodor counteractants, aroma therapy compositions, and combinations thereof, preferably said at least one active constituent is a fragrance. The composition may comprise between 0.1 wt. % and 5 wt. % of said at least one active constituent, based on the total weight of the composition, preferably between 0.5 wt. % ad 4 wt. %, more preferably between 1 wt. % and 3 wt. %, even more preferably between 1.5 wt. % and 2.5 wt. %.

The composition may then comprise between 0.1 wt. % and 5 wt. % of a fragrance, based on the total weight of the composition, preferably between 0.5 wt. % ad 4 wt. %, more preferably between 1 wt. % and 3 wt. %, even more preferably between 1.5 wt. % and 2.5 wt. %.

A wide variety of chemicals are known for perfumery, such as aldehydes, ketones, esters, alcohols, terpenes, and the like. Most conventional fragrance materials are volatile essential oils. A fragrance can be relatively simple in composition or can be a complex mixture of natural and synthetic chemical components.

Natural fragrances include naturally derived oils such as oil of Bergamot, Bitter Orange, Lemon, Mandarin, Caraway, Cedar Leaf, Clove Leaf, Cedar Wood, Geranium, Lavender, Orange, Origanum, Petitgrain, White Cedar, Patchouli, Lavandin, Neroli, Rose absolute, and the like. Natural perfumes include the extracts of blossoms, stems and leaves, fruits, fruit peel, roots, woods, herbs and grasses, needles and branches, resins and balsams. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetivert oil, olibanum oil, galbanum oil, ladanum oil and lavendin oil.

Typical synthetic perfume compounds are products of the ester, ether, aldehyde, ketone, alcohol and hydrocarbon type. Examples of perfume compounds of the ester type are benzyl acetate, p-tert.butyl cyclohexylacetate, linalyl acetate, phenyl ethyl acetate, linalyl benzoate, benzyl formate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, !Dial and bourgeonal. Examples of suitable ketones are the ionones and methyl cedryl ketone. Suitable alcohols are anethol, citronellol, eugenol, isoeugenol, geraniol, linalool, phenylethyl alcohol and terpineol. The hydrocarbons mainly include the terpenes and balsams.

Synthetic types of fragrance compositions either alone or in combination with natural oils are described in EP 2 810 663 B1.

The composition may be free of acetone.

Preferably the composition is an air freshener. As an air freshener, the composition makes the air in a room smell fresh. This means that the composition may comprise a fragrance but also an odor eliminator or an active constituent that has a double effect fragrance and odor elimination.

In a second aspect, the invention relates to a device comprising a container and a non-aerosol, spray generation dispenser, wherein the container contains the composition according to the invention and it attaches to the non-aerosol, spray generation dispenser.

The non-aerosol, spray generation dispenser sprays the composition according to the invention. The non-aerosol, spray generation dispenser may be an auto-actuable dispenser, which may be automatically actuated every 30 minutes, preferably every 20 minutes, more preferably every 10 minutes.

The non-aerosol, spray generation dispenser may be a manually-actuable dispenser.

It will be understood that the non-aerosol, spray generation dispenser may be an auto-actuable dispenser that can also be manually actuated.

The device is preferably an air-freshening or air-fragrancing device.

In a third aspect, the invention relates to a use of the composition according to the invention for freshening or fragrancing air.

The invention will be further described, by way of example, with the reference to the following non-limiting embodiments.

### EXAMPLES

Table 1 shows a composition according to the invention. This composition looks macroscopically like a single liquid phase, meaning that the composition is stable and that the fragrance was completely solubilized. Furthermore, when the composition is sprayed, it does not fallout and no residues are formed on surfaces; with other words, the sprayed composition is retained in the air.

TABLE 1
Composition according to the invention

| Ingredient | Wt. % |
|---|---|
| Isopropyl alcohol | 16 |
| Solketal (Augeo clean multi) | 30 |
| Fragrance | 2 |
| Water | 52 |

Hence one or more objects of the present invention are achieved by the present disclosure, which is further elucidated in the appended claims.

1. A composition comprising:
a. at least 10 wt. % of at least one C1 to C4 monohydric alcohol, based on the total weight of the composition;
b. at least 20 wt. % of at least one solvent selected from the group consisting of solketal, acetone, methylpropyl ketone, dipropyl ketone, dimethyl sulfoxide, glycerine carbonate, propylene carbonate, butylene carbonate, glycerine formal, 2-ethyl hexanol, 2-butyl octanol, 2- hexyl decanol, 2-octyl dodecanol, and mixtures thereof, based on the total weight of the composition;
c. between 0.001 wt. % and 5 wt. % of at least one active constituent, based on the total weight of the composition, wherein the active constituent is selected from the group consisting of fragrances, air-sanitisers, deodorizers, odor eliminators, malodor counteractants, insecticides, insect repellants, medicinal substances, disinfectants, aroma therapy compositions and combinations thereof; and
d. water.

2. The composition according to claim 1, wherein said composition is free of propellants.

3. The composition according to claim 1, wherein said at least one C1 to C4 monohydric alcohol is selected from the group consisting of ethanol, isopropyl alcohol and combinations thereof.

4. The composition according to claim 1, wherein said composition comprises at least 12 wt. % of said at least C1 to C4 monohydric alcohol, based on the total weight of the composition.

5. The composition according to claim 1, wherein said at least one solvent is solketal.

6. The composition according to claim 1, wherein said composition comprises at least 25 wt. % of said at least one solvent.

7. The composition according to claim 1, wherein the composition comprises less than 25 wt. % volatile organic compounds, based on the total weight of the composition.

8. The composition according to claim 1, wherein said at least one active constituent is selected from the group consisting of fragrances, deodorizers, odor eliminators, malodor counteractants, aroma therapy compositions, and combinations thereof.

9. The composition according to claim 1, where said composition comprises between 0.1 wt. % and 5 wt. % of said at least one active constituent, based on the total weight of the composition.

10. The composition according to claim 1, wherein said composition is free of acetone.

11. The composition according to claim 1, wherein said composition is an air freshener.

12. A device comprising a container and a non-aerosol, spray generation dispenser, wherein the container contains the composition according to claim 1 and it attaches to the non-aerosol, spray generation dispenser.

13. The device according to claim 12, wherein said non-aerosol, spray generation dispenser is an auto-actuable dispenser.

14. The device according to claim 13, wherein said non-aerosol, spray generation dispenser is automatically actuated every 30 minutes.

15. The device according to claim 12, wherein said non-aerosol, spray generation dispenser is a manually-actuable dispenser.

16. The device according to claim 12, wherein said device is an air-freshening or air-fragrancing device.

17. A method of freshening or fragrancing air, the method comprising:
dispensing the composition according to claim 1 into air.

18. The composition according to claim 3, wherein the at least one C1 to C4 monohydric alcohol is selected from the group consisting of ethanol, isopropyl alcohol and combinations thereof.

19. The composition according to claim 6, wherein said composition comprises between 25 wt. % and 50 wt. % of said at least one solvent.

20. The composition according to claim 7, wherein the composition comprises less than 20 wt. % volatile organic compounds, based on the total weight of the composition
